SEQUENCE LISTING (8 sequences)

SEQ ID NO: 1 - protein, 29 aa, Homo sapiens
HSQGTFTSDYSKYLDSRRAQDFVQWLMNT

SEQ ID NO: 2 - protein, 37 aa, Homo sapiens
HDEFERHAEGTFTSDVSSYLEGQAAKEFIAWLVKGRG

SEQ ID NO: 3 - protein, 33 aa, Homo sapiens
HADGSFSDEMNTILDNLAARDFINWLIQTKITD

SEQ ID NO: 4 - protein, 39 aa, Homo sapiens
HGEGTFTSDLSKQMEEEAVRLFIEWLKNGGPSSGAPPPS

SEQ ID NO: 5 - protein, 39 aa, Homo sapiens
HSDGTFTSDLSKQMEEEAVRLFIEWLKNGGPSSGAPPPS

SEQ ID NO: 6 - protein, 37 aa, Homo sapiens
HSQGTFTSDYSKYLDSRRAQDFVQWLMDTKRNKNNIA

SEQ ID NO: 7 - protein, 31 aa, Artificial Sequence
  gluocagon-like peptide
HAEGTFTSDVSSYLEGQAAKEFIAWLVRGRG

SEQ ID NO: 8 - protein, 33 aa, Artificial Sequence
  glucogon-like peptide
HGDGSFSDEMNTILDNLAARDFINWLIQTKITD

(19) United States
(12) Patent Application Publication
(10) Pub. No.: US 2024/0374690 A1
(43) Pub. Date: Nov. 14, 2024

(54) Pharmaceutical Peptide Compositions and Methods of Preparation Thereof

(71) Applicant: MYLAN IRELAND LIMITED, Dublin (IE)

(72) Inventors: Katarzyna Banasiak, Galway (IE); Neeraj sivadas, Galway (IE); Michael Paul Fitzgerald, Galway (IE); Claire Downes, Galway (IE); Paul Carr, Mayo (IE)

(21) Appl. No.: 18/262,535

(22) PCT Filed: Jan. 25, 2022
(86) PCT No.: PCT/IB2022/050644

§ 371 Date: Jul. 21, 2023

Related U.S. Application Data
(60) Provisional application No. 63/141,230, filed on Jan. 25, 2021.
(60) Provisional application No. 63/189,863, filed on May 18, 2021.
(60) Provisional application No. 63/226,020, filed on Jul. 27, 2021.

(51) Int. Cl. A61K 38/26; A61K 9/08; A61K 47/02; A61K 47/10; A61K 47/22; A61K 47/26; A61K 47/42

(52) U.S. Cl. CPC A61K 38/26; A61K 9/08; A61K 47/02; A61K 47/10; A61K 47/22; A61K 47/26; A61K 47/42

(57) ABSTRACT

Disclosed herein is a stabilized peptide composition that includes a glucagon-like peptide and a stabilizer. The stabilizer may be a stabilizing peptide having the same amino acid sequence as the glucagon-like peptide except the stabilizing peptide has at least one sequence alteration that may be a truncation, a deletion, a substitution and/or an insertion. Also disclosed are methods for making the stabilized peptide composition and its use for treating patients in need thereof.

This application claims priority to U.S. Provisional Application 63/141,230, filed Jan. 25, 2021, U.S. Provisional Application 63/189,863, filed May 18, 2021, and U.S. Provisional Application 63/226,020, filed Jul. 27, 2021, each of which is incorporated by reference herein in its entirety.

### SEQUENCING LISTING

The instant application contains a Sequence Listing which has been submitted in ASCII format via EFS-Web and is hereby incorporated by reference in its entirety. Said ASCII copy, created on May 18, 2021, is named “Sequence Listing.txt” and is 3,247 bytes in size.

### BACKGROUND

### Field of the Invention

The present disclosure relates to the field of pharmaceutical compositions. More specifically it pertains to methods for preparing stable pharmaceutical compositions which are prepared from a bulk peptide product that is stabilized via the addition of a stabilizing compound.

### Description of Related Art

Therapeutic peptides are widely used in medical practice. Pharmaceutical compositions of such therapeutic peptides are required to have a shelf life of several years in order to be suitable for common use. However, peptide compositions are inherently unstable due to sensitivity towards chemical and physical degradation. Chemical degradation involves change of covalent bonds, such as by oxidation, hydrolysis, racemization or crosslinking. Physical degradation involves conformational changes relative to the native structure of the peptide, i.e. secondary and tertiary structure, such as aggregation, precipitation or adsorption to surfaces.

Teduglutide is one therapeutic peptide that is a GLP-2 receptor analog used in patients with Short Bowel Syndrome (SBS) who are dependent on parenteral support (i.e., intravenous nutritional support). It is often dispensed to patients in a prefilled syringe or pen for a once daily subcutaneous injection. Prior to final formulation and packaging, the bulk drug product may be stored for a time period of a few weeks to a few months. Additionally, the reconstituted product is dispensed in an injection pen that may be stored for an extended period of time prior to use.

Liraglutide is another therapeutic peptide; it is a GLP-1 receptor agonist used in patients to assist in their management of type 2 diabetes mellitus and associated medical conditions. Similar to teduglutide, liraglutide is formulated as an aqueous solution, filtered and filled into cartridges. Those filled cartridges may be stored for several years prior to being administered to a patient. Thus, the aqueous solution in the cartridges must be stabile for an extended period of time.

US20090011976 discloses stabilized insulinotropic peptide and basal insulin compositions. The compositions includes poloxamer or polysorbate 20 surfactant as the stabilizing agent.

U.S. Pat. No. 7,998,927 discloses compositions and methods of increasing the stability and reducing the aggregation of peptides or proteins. The methods comprises adding an alkylglycoside or saccharide alkyl ester surfactant to the peptide or protein.

U.S. Pat. No. 8,114,959 discloses a method for increasing the shelf-life of a pharmaceutical composition that comprises a glucagon-like peptide. The method comprises drying, preferably freeze drying, the peptide product at a pH above neutral pH.

WO 2014096440 discloses a stabilized composition of GLP-2, or an analog thereof. The composition uses highly purified human serum albumin as the stabilizing agent.

WO 2019086559 discloses a stabilized composition of GLP-2, or an analog thereof. The composition uses a co-polyamino acid having carboxylate charges and hydrophobic radicals as the stabilizing agent in order to prevent DPP-IV degradation of the polypeptide.

U.S. Pat. No. 8,222,205 discloses a method by which an inactive GLP-1 compound can be converted into an active GLP-1 compound. The method comprises stirring the inactive compound in base (or acid) for a short period of time followed by neutralization and lyophilization. This method is carried out in a way where substantially no racemization of any of the amino acids occurs.

US20100056451 discloses a method for increasing the shelf-life of a pharmaceutical composition that comprises a glucagon-like peptide. The method comprises treating the peptide at a pH above neutral pH.

Degradation is a potential problem that can occur throughout the shelf life of a product when stored in the final packaging. This degradation of an active therapeutic peptide can create difficulties for both the patient and manufacturer. Because degradation must be reduced or eliminated in the final packaged product that may be stored for several years, there is a need to provide a stabilized therapeutic peptide composition that can be stored for an extended period of time in the final packaging.

### SUMMARY

Disclosed herein is an aqueous stabilized peptide composition. The composition comprises a glucagon-like peptide and a stabilizer (alternatively called a stabilizing agent).

Without being bound by a specific theory, it is thought that teduglutide consists an alpha-helical chain as shown in FIG. 8. During fibrillogenesis (one known degradation pathway), the molecule unfolds exposing hydrophobic residues. The alpha-helix converts to beta-sheet. Two beta-strands per molecule link to each other through hydrophobic interactions. Fibril formation occurs when the teduglutide monomers (each containing two beta-strands) are stacked together via hydrogen bonds. Thus, a method by which to either reduce unfolding of the alpha helix and/or prevent beta sheet stacking should reduce or prevent fibrillogenesis and prolong the shelf-life of teduglutide. It is thought that the inclusion of a stabilizing agent can reduce and/or prevent fibrillogenesis.

Without being bound by a specific theory, it is thought that liraglutide consists of two alpha-helical segments as shown in FIG. 1. Amino acids involved in the two helices are noted in the two boxes. During fibrillogenesis, the molecule unfolds exposing hydrophobic residues. The alpha-helices convert to beta-strands. The two beta-strands per molecule link to each other through hydrophobic interactions. Fibril formation occurs when the liraglutide monomers (each containing the 2 beta-strands above) are stacked together via hydrogen bonds. Thus, a method by which to either reduce unfolding of the alpha helix and/or prevent beta sheet stacking should reduce or prevent fibrillogenesis and prolong the shelf-life of liraglutide. It is thought that the inclusion of a stabilizing agent can reduce and/or prevent fibrillogenesis.

Also disclosed herein is an aqueous stabilized peptide composition. The composition comprises 10 mg/mL of teduglutide, 6.87 mg/mL of dibasic sodium phosphate heptahydrate, 1.29 mg/mL of monobasic sodium phosphate monohydrate, 7.76 mg/mL of L-histidine, 30 mg/mL of mannitol, and 4% w/w relative to teduglutide, a stabilizing peptide that has the same amino acid sequence as teduglutide wherein said stabilizing peptide comprises a three amino acid truncation on the C-terminus.

Also disclosed herein is an aqueous stabilized peptide composition. The composition comprises 6 mg/mL of liraglutide, 1.42 mg/mL of sodium hydrogen phosphate dihydrate, 14 mg/mL of propylene glycol, 5.5 mg/mL of phenol, and 2% w/w, relative to liraglutide, a stabilizing peptide that has the same amino acid sequence as liraglutide wherein said stabilizing peptide has a Ser¹⁴ to D-Ser¹⁴ substitution in the amino acid sequence.

Clause 1. An aqueous stabilized peptide composition, said composition comprising a glucagon-like peptide, and a stabilizer.

Clause 2. The aqueous stabilized peptide composition according to clause 1, wherein the glucagon-like peptide is selected from group consisting of glucagon, a glucagon analogue, a glucagon derivative, oxynthomodulin, GLP-1, a GLP-1 analogue, a derivative of GLP-1, a derivative of a GLP-1 analogue, GLP-2, a GLP-2 analogue, a derivative of GLP-2, a derivative of a GLP-2 analogue, exendin-4, an exendin-4 analogue, a derivative of exendin-4, and a derivative of an exendin-4 analogue.

Clause 3. The aqueous stabilized peptide composition according to any of the previous clauses, wherein the glucagon-like peptide is selected from the group consisting of GLP-2 a GLP-2 analogue, a derivative of GLP-2, and a derivative of a GLP-2 analogue.

Clause 4. The aqueous stabilized peptide composition according to clause 3, wherein the glucagon-like peptide is teduglutide.

Clause 5. The aqueous stabilized peptide composition according to any of clauses 1 or 2, wherein the glucagon-like peptide is selected from the group consisting of GLP-1, a GLP-1 analogue, a derivative of GLP-1, and a derivative of a GLP-1 analogue.

Clause 6. The aqueous stabilized peptide composition according to claim 5, wherein the glucagon-like peptide is liraglutide.

Clause 7. The aqueous stabilized peptide composition according to any of the previous clauses, wherein the aqueous stabilized peptide composition further comprises a buffer selected from the group consisting of phosphate, TRIS, PBS, glycine, N-glycylglycine, sodium acetate, sodium carbonate, glycylglycine, histidine, lysine, arginine, sodium citrate and combinations thereof.

Clause 8. The aqueous stabilized peptide composition according to clause 7, wherein the buffer comprises phosphate.

Clause 9. The aqueous stabilized peptide composition according to clause 8, wherein the phosphate buffer has a pH in a range of from 6.0 to 12.0.

Clause 10. The aqueous stabilized peptide composition according to any of the previous clauses, wherein the aqueous stabilized peptide composition further comprises an isotonicity agent selected from the group consisting of sodium chloride, xylitol, mannitol, sorbitol, glycerol, glucose, maltose, sucrose, L-glycine, L-histidine, arginine, lysine, isoleucine, aspartic acid, tryptophan, threonine, dimethyl sulfone, polyethylene glycol, propylene glycol and combinations thereof.

Clause 11. The aqueous stabilized peptide composition according to clause 10, wherein the isotonicity agent is mannitol or propylene glycol.

Clause 12. The aqueous stabilized peptide composition according to any of the previous clauses, wherein the aqueous stabilized peptide composition further comprises a stabilizer selected from the group consisting of polyvinyl pyrrolidone, polyvinyl alcohol-polyethylene glycol, Pluronic F68, tocopherol polyethylene glycol succinate, hydroxypropyl methylcellulose, hydroxyethyl cellulose, hydroxypropyl cellulose, methylcellulose, carboxymethylcellulose sodium, polyvinyl alcohol, sodium alginate, Tween 80, an amino acid (e.g., L-histidine), and a stabilizing peptide, surfactants, xanthan gum, acacia, and combinations thereof.

Clause 13. The aqueous stabilized peptide composition according to clause 12, wherein the stabilizer is L-histidine.

Clause 14. The aqueous stabilized peptide composition according to clause 12, wherein the stabilizer is a stabilizing peptide, wherein said stabilizing peptide has the same amino acid sequence as the glucagon-like peptide wherein the stabilizing peptide comprises at least one sequence alternation when compared to the glucagon-like peptide.

Clause 15. The aqueous stabilized peptide composition according to any of the previous clauses, wherein the aqueous stabilized peptide composition further comprises a preservative selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thimerosal, and combinations thereof.

Clause 16. The aqueous stabilized peptide composition according to clause 15, wherein the preservative is phenol.

Clause 17. An aqueous stabilized peptide composition, said composition comprising 10 mg/mL of teduglutide, 6.87 mg/mL of dibasic sodium phosphate heptahydrate, 1.29 mg/mL of monobasic sodium phosphate monohydrate, 7.76 mg/mL of L-histidine, 30 mg/ml of mannitol, and 4% w/w, relative to teduglutide, a stabilizing peptide that has the same amino acid sequence as teduglutide wherein said stabilizing peptide has a three amino acid truncation on the C-terminus.

Clause 18. A method for making the stabilized peptide composition according to clause 17, said method comprising: a) dissolving the dibasic sodium phosphate heptahydrate and monobasic sodium phosphate monohydrate in water to make Solution A; b) adding the stabilizing peptide to Solution A to make Solution B; c) adding the teduglutide to Solution B to make Solution C; d) dissolving the L-histidine and mannitol in water to make Solution D; c) combining Solution D and Solution C to make the Combined Solution; and f) adjusting the pH of the Combined Solution to between 6.0 and 9.0 using either HCl(aq) or NaOH(aq).

Clause 19. An aqueous stabilized peptide composition, said composition comprising 6 mg/mL of liraglutide, 1.42 mg/mL of sodium hydrogen phosphate dihydrate, 14 mg/mL of propylene glycol, 5.5 mg/mL of phenol, and 2% w/w, relative to liraglutide, a stabilizing peptide that has the same amino acid sequence as liraglutide wherein said stabilizing peptide has a Ser14 to D-Ser14 substitution in the amino acid sequence.

Clause 20. A method for making the stabilized peptide composition according to clause 19, said method comprising: a) dissolving the sodium hydrogen phosphate dihydrate, propylene glycol and phenol in water to make Solution A; b) dissolving the liraglutide in water to make Solution B; c) dissolving the stabilizing peptide in water to make Solution C; d) combining Solution A, Solution B and Solution C while mixing to make the Combined Solution; and e) adjusting the pH of the Combined Solution to between 8.1 and 8.2 using either HCl(aq) or NaOH(aq).

Clause 21. The method according to clause 18 or clause 20 further comprising adding water to the Combined Solution.

Clause 22. The method according to clause 18 or clause 20, wherein said glucagon-like peptide and stabilizing peptide are treated with base before preparing said stabilized peptide composition, wherein the stabilizer is a stabilizing peptide, wherein treating with base comprises combining the glucagon-like peptide and stabilizing peptide in an aqueous solution of base, stirring or agitating the aqueous solution for from 10 minutes to 6 hours, and lyophilizing the aqueous solution comprising the glucagon-like peptide, stabilizing peptide and base.

Clause 23. The aqueous stabilized peptide composition according to clause 1, wherein the glucagon-like peptide and/or the stabilizer are treated with base before incorporation into the stabilized peptide composition.

### BRIEF DESCRIPTION OF THE DRAWING(S)

FIG. 1 illustrates the structure and amino acid sequence of teduglutide. The amino acids in the red boxes are thought to be involved in amyloid formation via fibrillogenesis.

FIG. 2 illustrates the transformation of teduglutide from the alpha helix to a beta-sheet which is postulated to be the primary mechanism for amyloid formation.

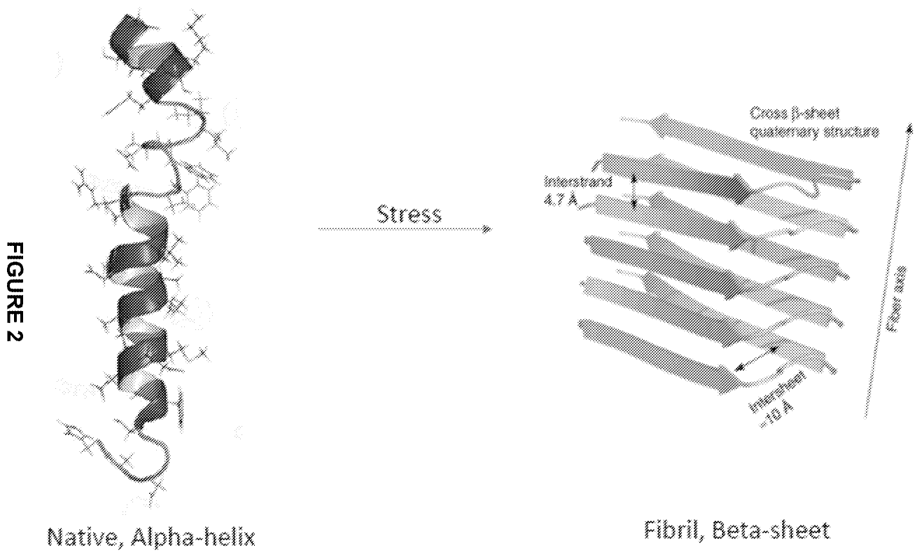

FIG. 3 lists the amino acid sequence and numbering of several notable peptides.

FIG. 4 depicts ThT assay results for one teduglutide composition comprising a stabilizing peptide.

FIG. 5 depicts the disruption of fibril seed formation when a protein is treated with base.

FIG. 6 depicts ThT assay results for one composition where the teduglutide was subject to treatment with base prior to formulation.

FIG. 7 depicts ThT assay results for one composition where the teduglutide was subject to treatment with base for different time periods.

FIG. 8 illustrates the structure and amino acid sequence of liraglutide.

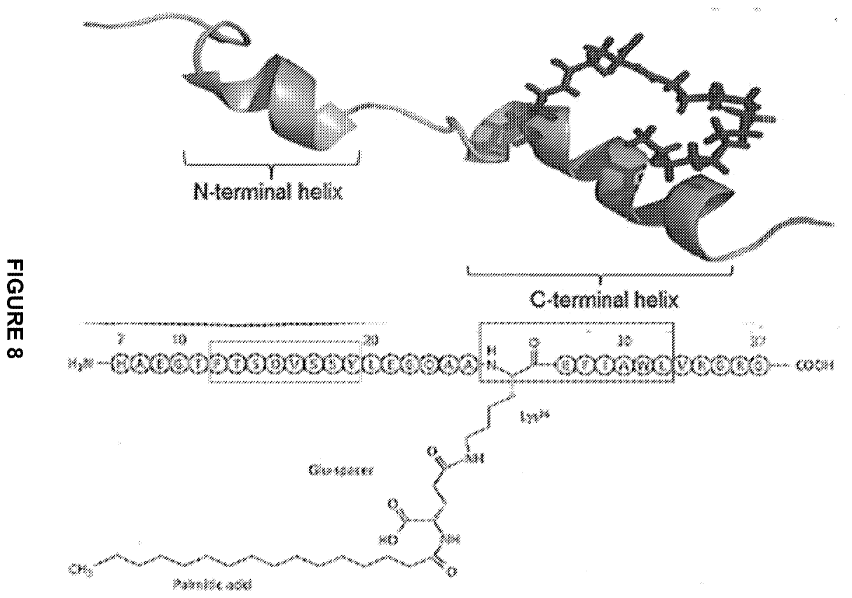

FIG. 9 depicts ThT assay results for several liraglutide solutions comprising a stabilizing peptide.

FIG. 10 depicts ThT assay results for several liraglutide solutions comprising a stabilizing peptide.

### DEFINITIONS

The following are detailed definitions of specific terms used in the specification.

When introducing elements of the present disclosure or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

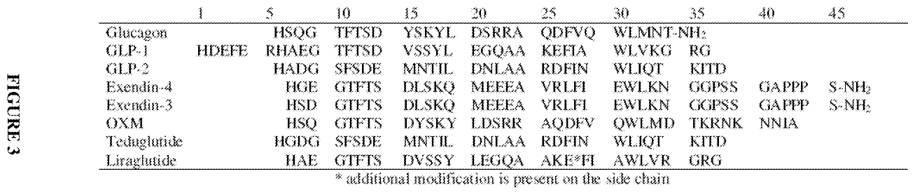

The term “glucagon-like peptide” as used herein refers to the homologous peptides derived from the preproglucagon gene, exendins and analogues and derivatives thereof. The peptides derived from the preproglucagon gene are glucagon, glucagon-like peptide 1 (GLP-1), glucagon-like peptide 2 (GLP-2) and oxynthomodulin (OXM). The exendins which are found in the Gila monster are homologous to GLP-1 and also exert an insulinotropic effect. Examples of exendins are exendin-4 and exendin-3. Notable glucagon-like peptides have amino acid sequences shown in FIG. 3 and in the Sequence Listing included herewith.

The term “analogue” as used herein in reference to a peptide means a modified peptide wherein one or more amino acid residues of the peptide have been substituted by other amino acid residues and/or wherein one or more amino acid residues have been deleted from the peptide and/or wherein one or more amino acid residues have been conformationally altered from the peptide and/or wherein one or more amino acid residues have been added to the peptide. Such addition or deletion of amino acid residues can take place at the N-terminal of the peptide and/or at the C-terminal of the peptide. Two different and simple systems are often used to describe analogues: For example Arg³⁴-GLP-1(7-37) or K³⁴R-GLP-1(7-37) designates a GLP-1 analogue wherein amino acid residues at position 1-6 have been deleted, and the naturally occurring lysine at position 34 has been substituted with arginine (standard single letter abbreviation for amino acids used according to IUPAC-IUB nomenclature).

The term “derivative” as used herein in relation to a parent peptide means a chemically modified parent protein or an analogue thereof, wherein at least one substituent is not present in the parent protein or an analogue thereof, i.e. a parent protein which has been covalently modified. Typical modifications are amides, carbohydrates, alkyl groups, acyl groups, esters, pegylations and the like. An examples of a derivative of GLP-1(7-37) is Arg³⁴, Lys²⁶(N^{ε}-(γ-Glu(N^{α}-hexadecanoyl)))-GLP-1(7-37) which designates a GLP-1 analogue where amino acid residues at positions 1-6 have been deleted, the naturally occurring lysine at position 26 has been functionalized with an N^{ε}-(γ-Glu(Na-hexadecanoyl) moiety.

The term “GLP-1 peptide” as used herein means GLP-1(7-37), a GLP-1 analogue, a GLP-1 derivative or a derivative of a GLP-1 analogue.

The term “GLP-2 peptide” as used herein means GLP-2(1-33), a GLP-2 analogue, a GLP-2 derivative or a derivative of a GLP-2 analogue.

The term “exendin-4 peptide” as used herein means exendin-4(1-39), an exendin-4 analogue, an exendin-4 derivative or a derivative of an exendin-4 analogue.

The term “bulk product” or “bulk peptide product” as used herein means the purified peptide product which is to be used for the manufacture of a pharmaceutical composition. Thus, the bulk product is normally obtained as the product from the final purification, drying or conditioning step. The bulk product may be crystals, precipitate, solution or suspension. The bulk product is also known in the art as the drug substance.

The term “therapeutically effective amount” as used herein means a dosage which is sufficient to be effective for the treatment of the patient compared with no treatment.

The term “treatment” or “treat” with respect to a disease or medical condition as used herein means the management and care of a patient having developed a disease, condition or disorder. The purpose of treatment is to combat the disease, condition or disorder. Treatment includes the administration of the pharmaceutical composition described herein to alleviate one or more symptoms associated with the disease, medical condition or disorder. Treatment may result in the alleviation of all symptoms or curing of said disease, medical condition or disorder.

The term “sequence alteration” with respect to a peptide or protein means that the amino acid sequence includes one more of a truncation, a deletion, a substitution or an insertion. A truncation means that one or more amino acids have been removed from the C-terminus and/or the N-terminus of the peptide or protein. A deletion means that one or more amino acids have been removed from the peptide or protein, and that removal is not on the C-terminus or the N-terminus (i.e., it is different from a truncation). A substitution means that one or more amino acids in the peptide or protein has been replaced by another amino acid. Said replacement amino acid is not limited. Non-limiting examples include a D-amino acid, a beta-amino acid, a naturally occurring amino acid, and a non-naturally occurring amino acid. An insertion means that one or more amino acids are added to the amino acid sequence of the peptide or protein. The insertion can be anywhere along the sequence including the C-terminus or the N-terminus. Said inserted amino acid is not limited. Non-limiting examples include a D-amino acid, a beta-amino acid, a naturally occurring amino acid, and a non-naturally occurring amino acid. In some instances, the peptide or protein has more than one sequence alteration where each alteration is independently selected from a truncation, a deletion, a substitution or an insertion.

### DETAILED DESCRIPTION

The examples and embodiments included herein are illustrations only. A person skilled in the art will recognize that alternative equivalent embodiments are also within the scope of the subject matter herein.

For all numerical values and ranges cited herein, they should be construed as being within about ±10% of the numerical value. Thus, for example, if a concentration is cited as being 2 mg/mL, it should be construed to mean from 1.8 to 2.2 mg/mL inclusive.

Disclosed herein is stabilized peptide composition (also called a pharmaceutical composition), methods of preparing said composition, and methods of treating patients in need of treatment with said composition. The composition comprises at least one glucagon-like peptide and at least one stabilizer. In some aspects, the stabilizer is a stabilizing peptide. In some aspects, the stabilized peptide composition is an aqueous composition. In some aspects, the composition further comprises one or more pharmaceutically acceptable excipients. For example, in some embodiments, the composition further comprises one or more of a buffer, an isotonicity agent, a preservative, and/or a stabilizer.

In some embodiments said glucagon-like peptide is glucagon, a glucagon analogue or a derivative thereof.

In another embodiment said glucagon-like peptide is oxynthomodulin.

In another embodiment said glucagon-like peptide is GLP-1, a GLP-1 analogue, a derivative of GLP-1 or a derivative of a GLP-1 analogue.

In another embodiment said GLP-1 analogue is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), and analogues thereof. In another embodiment said derivative of a GLP-1 analogue is liraglutide which has a sequence of Arg³⁴, Lys²⁶ (N^{ε}-(γ-Glu(N^{α}-hexadecanoyl)))-GLP-1(7-37).

Methods for the preparation of GLP-1, analogues thereof as well as GLP-1 derivatives can be found in e.g. WO 99/43706, WO 00/55119, WO 00/34331 and WO 03/18516.

In another embodiment the glucagon-like peptide is a GLP-1 peptide and the pharmaceutical composition, or a reconstituted composition thereof, has a glucagon-like peptide concentration from 0.1 mg/mL to 50 mg/mL, from 0.1 mg/mL to 25 mg/mL, from 1 mg/mL to 25 mg/mL, from 1 mg/mL to 10 mg/mL, or from 3 mg/mL to 8 mg/mL. In another embodiment the GLP-1, a GLP-1 analogue, a derivative of GLP-1 or derivative of a GLP-1 analogue has a concentration of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL.

In another embodiment the glucagon-like peptide is GLP-2, a GLP-2 analogue, a derivative of GLP-2 or a derivative of a GLP-2 analogue.

In another embodiment the derivative of GLP-2, or a derivative of a GLP-2 analogue, has a lysine residue, such as one lysine, wherein a lipophilic substituent optionally via a spacer is attached to the epsilon amino group of said lysine. In another embodiment the derivative of GLP-2, or a derivative of a GLP-2 analogue, is teduglutide.

Methods for the preparation of GLP-2, analogs thereof as well as GLP-2 derivatives can be found in e.g. WO 99/43361, WO 00/55119, U.S. Pat. Nos. 5,789,379, 5,834,428, 6,184,201, US 2003/0162703, and US 2006/0105954.

In another embodiment the glucagon-like peptide is a GLP-2 peptide and the pharmaceutical composition, or a reconstituted composition thereof, has a glucagon-like peptide concentration from 0.1 mg/mL to 100 mg/mL, from 0.1 mg/mL to 25 mg/mL, or from 1 mg/mL to 25 mg/mL. In another embodiment the GLP-2, a GLP-2 analogue, a derivative of GLP-2 or derivative of a GLP-2 analogue has a concentration of 1 mg/mL, 2 mg/mL, 3 mg/mL, 4 mg/mL. 5 mg/mL, 6 mg/mL, 7 mg/mL, 8 mg/mL, 9 mg/mL, 10 mg/mL. 11 mg/mL, 12 mg/mL. 13 mg/mL, 14 mg/mL, 15 mg/mL, 16 mg/mL, 17 mg/mL, 18 mg/mL, 19 mg/mL, 20 mg/mL.

In another embodiment the glucagon-like peptide is exendin-4, an exendin-4 analogue, a derivative of exendin-4, or a derivative of an exendin-4 analogue.

In another embodiment the glucagon-like peptide is exendin-4. In another embodiment the glucagon-like is peptide ZP-10 ([Ser³⁸Lys³⁹]exendin-4(1-39)LysLysLysLysLys-NH₂).

Methods for the preparation of exendin-4, analogues thereof, as well as exendin-4 derivatives can be found in e.g. WO 99/43708, WO 00/41546 and WO 00/55119.

In another embodiment the glucagon-like peptide is an exendin-4 peptide and the pharmaceutical composition, or a reconstituted composition thereof, has a concentration of glucagon-like peptide from 5 μg/mL to 10 mg/mL, from 5 μg/mL to 5 mg/mL, from 5 μg/mL to 5 mg/mL, from 0.1 mg/mL to 3 mg/mL, or from 0.2 mg/mL to 1 mg/mL. In some embodiments the glucagon-like peptide is exendin-4, an exendin-4 analogue, a derivative of exendin-4, or derivative of an exendin-4 analogue has a concentration of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL. 19 mg/mL. 20 mg/mL.

Buffers suitable for use in the pharmaceutical composition include, but are not limited to, phosphate, TRIS, PBS, glycine, N-glycylglycine, citrate sodium acetate, sodium carbonate, glycylglycine, histidine, lysine, arginine, and sodium citrate and combinations thereof. In another embodiment the pharmaceutical composition comprises a Tris buffer. In another embodiment the pharmaceutical composition comprises a Bicine buffer. In another embodiment the pharmaceutical composition comprises a phosphate buffer. For a phosphate buffer, any phosphate salt or combination of salts are used such that the pH of the composition is as described elsewhere herein. For example, one or more of H₃PO₄, H₂PO₄⁻, HPO₄²⁻ and PO₄³⁻ are combined in amounts such that the pH of the composition is as described elsewhere herein. The counterion ion can be any pharmaceutically acceptable counterion. Examples include, but are not limited to, sodium, potassium, calcium and magnesium.

The concentration of the buffer is selected based on the manner in which the composition is administered to patients. For example, pharmaceutically acceptable buffers, when part of a composition for parenteral administration, must have specific tonicity and concentrations in order to be most effectively administered. In all aspects, the concentration of the buffer will be such that it is safe for administration to human patients. In some embodiments, the concentration of the buffer is from about 10⁻³ M to about 2.0 M. In some embodiments, the concentration of the buffer is from about 10⁻² M to about 1.5 M. In some embodiments, the concentration of the buffer is from about 10⁻¹ M to about 1.0 M. Alternatively, in some embodiments, the concentration of each component of the buffer is between 0.1 and 10.0 mg/mL. In some embodiments, the concentration of each component of the buffer is between 0.5 and 7.0 mg/mL. In some embodiments, the concentration of each component of the buffer is between 1.0 and 7.0 mg/mL. In some embodiments, the concentration of each component of the buffer is between 1.0 and 2.0 mg/mL. In some embodiments, the concentration of each component of the buffer is between 1.25 and 1.75 mg/mL.

In another embodiment the pharmaceutical composition has a pH in the range from 5.0 to 10.0. In another embodiment the pharmaceutical composition has a pH in the range from 5.5 to 9.5. In another embodiment the pharmaceutical composition has a pH in the range from 6.0 to 9.0. In another embodiment the pharmaceutical composition has a pH in the range from 6.5 to 8.5. In another embodiment the pharmaceutical composition has a pH in the range from 7.0 to 8.0. In another embodiment the pharmaceutical composition has a pH in the range from 7.5 to 12.0. In another embodiment the pharmaceutical composition has a pH in the range from 7.5 to 11.5. In another embodiment the pharmaceutical composition has a pH in the range from 7.5 to 11.0. In another embodiment the pharmaceutical composition has a pH in the range from 8.1 to 10.7. In another embodiment the pharmaceutical composition has a pH in the range from 8.5 to 12.0. In another embodiment the pharmaceutical composition has a pH in the range from 8.5 to 11.5. In another embodiment the pharmaceutical composition has a pH in the range from 8.5 to 11.0. In another embodiment the pharmaceutical composition has a pH in the range from 8.5 to 10.7. In another embodiment the pharmaceutical composition has a pH in the range from 8.5 to 10.5. In another embodiment the pharmaceutical composition has a pH in the range from 9.0 to 12.5. In another embodiment the pharmaceutical composition has a pH in the range from 9.0 to 12.0. In another embodiment the pharmaceutical composition has a pH in the range from 9.0 to 11.5. In another embodiment the pharmaceutical composition has a pH in the range from 9.0 to 11.0. In another embodiment the pharmaceutical composition has a pH in the range from 9.0 to 10.7. In another embodiment the pharmaceutical composition has a pH in the range from 9.0 to 10.5. In another embodiment the pharmaceutical composition has a pH in the range from 8.5 to 10.0. In another embodiment the pharmaceutical composition has a pH in the range from 8.5 to 9.6. In another embodiment the pharmaceutical composition has a pH in the range from 7.5 to 9.5. In another embodiment the pharmaceutical composition has a pH in the range from 7.9 to 8.4. In another embodiment the pharmaceutical composition has a pH in the range from 8.1 to 8.2.

In another embodiment the pharmaceutical composition has a pH of about 7.0. In another embodiment the pharmaceutical composition has a pH of about 7.1. In another embodiment the pharmaceutical composition has a pH of about 7.2. In another embodiment the pharmaceutical composition has a pH of about 7.3. In another embodiment the pharmaceutical composition has a pH of about 7.4. In another embodiment the pharmaceutical composition has a pH of about 7.5. In another embodiment the pharmaceutical composition has a pH of about 7.6. In another embodiment the pharmaceutical composition has a pH of about 7.7. In another embodiment the pharmaceutical composition has a pH of about 7.8. In another embodiment the pharmaceutical composition has a pH of about 7.9. In another embodiment the pharmaceutical composition has a pH of about 8.0.

The pH of the pharmaceutical composition is set by use of a buffer as described elsewhere herein. In some aspects, the pH of the pharmaceutical composition is adjusted by the use of either a pharmaceutically acceptable aqueous acid or base as needed. In some embodiments the pharmaceutically acceptable aqueous acid is HCl(aq). In some embodiments the pharmaceutically acceptable aqueous base is NaOH(aq).

In some embodiments, the pharmaceutical composition comprises one or more preservatives. Preservatives for use in the compositions herein include, but are not limited to, phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thimerosal, and combinations thereof. In some embodiments, the preservative is phenol.

In some embodiments, the preservative has a concentration of from 1.0 to 50 mg/ml in the composition. In some embodiments, the preservative has a concentration of from 1.0 to 50 mg/mL in the composition. In some embodiments, the preservative has a concentration of from 2.0 to 40 mg/mL in the composition. In some embodiments, the preservative has a concentration of from 3.0 to 30 mg/mL in the composition. In some embodiments, the preservative has a concentration of from 4.0 to 20 mg/mL in the composition. In some embodiments, the preservative has a concentration of from 5.0 to 10 mg/mL in the composition. In some embodiments the preservative has a concentration of 1 mg/mL. 1.5 mg/mL, 2 mg/mL. 2.5 mg/mL. 3 mg/mL. 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL. 9.5 mg/mL, 10 mg/mL.

In some embodiments the pharmaceutical composition herein comprises one or more isotonicity agents. Isotonicity agents include, but are not limited to, sodium chloride, xylitol, mannitol, sorbitol, glycerol, glucose, maltose, sucrose, L-glycine, L-histidine, arginine, lysine, isoleucine, aspartic acid, tryptophan, threonine, dimethyl sulfone, polyethylene glycol, propylene glycol and combinations thereof.

In some embodiments, the isotonicity agent has a concentration of from 1.0 to 50 mg/mL in the composition. In some embodiments, the isotonicity agent has a concentration of from 2.0 to 45 mg/mL in the composition. In some embodiments, the isotonicity agent has a concentration of from 5.0 to 40 mg/mL in the composition. In some embodiments, the isotonicity agent has a concentration of from 10 to 35 mg/mL in the composition. In some embodiments, the isotonicity agent has a concentration of from 15 to 35 mg/mL in the composition. In some embodiments, the isotonicity agent has a concentration of from 20 to 35 mg/mL in the composition. In some embodiments, the isotonicity agent has a concentration of from 3.0 to 30 mg/mL in the composition. In some embodiments, the isotonicity agent has a concentration of from 4.0 to 20 mg/mL in the composition. In some embodiments, the isotonicity agent has a concentration of from 5.0 to 15 mg/mL in the composition. In some embodiments, the isotonicity agent has a concentration of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL. 29 mg/mL. 30 mg/mL, 31 mg/mL, 32 mg/mL, 33 mg/mL, 34 mg/mL, 35 mg/mL. 36 mg/mL, 37 mg/mL, 38 mg/mL, 39 mg/mL, 40 mg/mL.

In some embodiments, the pharmaceutical composition comprises one or more stabilizers. Stabilizers include, but are not limited to, polyvinyl pyrrolidone, polyvinyl alcohol-polyethylene glycol, Pluronic F68, tocopherol polyethylene glycol succinate, hydroxypropyl methylcellulose, hydroxyethyl cellulose, hydroxypropyl cellulose, methylcellulose, carboxymethylcellulose sodium, polyvinyl alcohol, sodium alginate, Tween 80, an amino acid (e.g., L-histidine), a stabilizing peptide, surfactants, xanthan gum, acacia, and combinations thereof. In some embodiments, the stabilizing agent is L-histidine. In some embodiments, the stabilizing agent is a stabilizing peptide.

As used herein, the term ‘stabilizing peptide’ refers to a poly(peptide) comprising two or more amino acids. The sequence of said amino acids is as described herein. In some embodiments the pharmaceutical composition comprises one or more stabilizing peptides. In some aspects, the stabilizing peptide is a glucagon-like peptide that comprises at least one sequence alteration. For example, in an embodiment where the glucagon-like peptide is GLP-2, the natural amino acid sequence may comprise a truncation from either terminus or it may comprise a substitution. Any sequence alteration in the glucagon-like peptide is acceptable such that the pharmaceutical composition is stabilized for a time period longer as compared to if the stabilizing peptide had not been added to the composition. In some embodiments, the stabilizing peptide has the same amino acid sequence as the glucagon-like peptide except for the one or more sequence alterations. In some embodiments, the stabilizing peptide has a different amino acid sequence as compared to the glucagon-like peptide.

In some aspects, the stabilizing peptide is a glucagon-like peptide where one or more of the naturally occurring amino acids is replaced with the opposite stereoisomer. For example, in an embodiment where the glucagon-like peptide is GLP-1, the natural amino acid (i.e., the L isomer) Ser¹⁰ is replaced with D-Ser¹⁰. Any single amino acid in the glucagon-like peptide can be replaced by its opposite enantiomer such that the pharmaceutical composition is stabilized for a time period longer as compared to if the stabilizing peptide had not been added to the composition. In some embodiments, the stabilizing peptide has the same amino acid sequence as the glucagon-like peptide except for the one or more L-amino acids that are replaced by D-amino acids. In some embodiments, the stabilizing peptide has a different amino acid sequence as compared to the glucagon-like peptide.

In some embodiments the pharmaceutical composition comprises GLP-1, a GLP-1 analogue, a derivative of GLP-1 or a derivative of a GLP-1 analogue, and the stabilizing peptide comprises GLP-1, a GLP-1 analogue, a derivative of GLP-1 or a derivative of a GLP-1 analogue that comprises at least one sequence alteration.

In some embodiments the pharmaceutical composition comprises GLP-1, a GLP-1 analogue, a derivative of GLP-1 or a derivative of a GLP-1 analogue, and the stabilizing peptide comprises GLP-1, a GLP-1 analogue, a derivative of GLP-1 or a derivative of a GLP-1 analogue where one or more of the L-amino acids has been replaced with a D-amino acid having the same side chain. In some embodiments, only one L-amino acid replaced with a D-amino acid.

In some embodiments, the glucagon-like peptide is liraglutide which differs from GLP-1 by having an arginine at position 34 in the amino acid sequence and an additional modification attached to the sidechain of Lys²⁶. The amino acid sequence of liraglutide is shown elsewhere herein. In some embodiments, the stabilizing peptide has the same amino acid sequence as liraglutide except one or more of the L-amino acids is substituted with a D-amino acid having the same side chain.

In some embodiments, the stabilizing peptide has the same amino acid sequence as liraglutide except that His⁷ is replaced with D-His⁷. In some embodiments, the stabilizing peptide has the same amino acid sequence as liraglutide except that Ala⁸ is replaced with D-Ala⁸. In some embodiments, the stabilizing peptide has the same amino acid sequence as liraglutide except that Glu⁹ is replaced with D-Glu⁹. In some embodiments, the stabilizing peptide has the same amino acid sequence as liraglutide except that Thr¹¹ is replaced with D-Thr¹¹. In some embodiments, the stabilizing peptide has the same amino acid sequence as liraglutide except that Phe¹² is replaced with D-Phe¹². In some embodiments, the stabilizing peptide has the same amino acid sequence as liraglutide except that Thr¹³ is replaced with D-Thr¹³. In some embodiments, the stabilizing peptide has the same amino acid sequence as liraglutide except that Ser¹⁴ is replaced with D-Ser¹⁴. In some embodiments, the stabilizing peptide has the same amino acid sequence as liraglutide except that Asp¹⁵ is replaced with D-Asp¹⁵. In some embodiments, the stabilizing peptide has the same amino acid sequence as liraglutide except that Val¹⁶ is replaced with D-Val¹⁶. In some embodiments, the stabilizing peptide has the same amino acid sequence as liraglutide except that Ser¹⁷ is replaced with D-Ser¹⁷. In some embodiments, the stabilizing peptide has the same amino acid sequence as liraglutide except that Ser¹⁸ is replaced with D-Ser¹⁸. In some embodiments, the stabilizing peptide has the same amino acid sequence as liraglutide except that Tyr¹⁹ is replaced with D-Tyr¹⁹. In some embodiments, the stabilizing peptide has the same amino acid sequence as liraglutide except that Leu²⁰ is replaced with D-Leu²⁰. In some embodiments, the stabilizing peptide has the same amino acid sequence as liraglutide except that Glu²¹ is replaced with D-Glu²¹. In some embodiments, the stabilizing peptide has the same amino acid sequence as liraglutide except that Gln²³ is replaced with D-Gln²³. In some embodiments, the stabilizing peptide has the same amino acid sequence as liraglutide except that Ala²⁴ is replaced with D-Ala²⁴. In some embodiments, the stabilizing peptide has the same amino acid sequence as liraglutide except that Ala²⁵ is replaced with D-Ala²⁵. In some embodiments, the stabilizing peptide has the same amino acid sequence as liraglutide except that Lys²⁶ is replaced with D-Lys²⁶. In some embodiments, the stabilizing peptide has the same amino acid sequence as liraglutide except that Glu²⁷ is replaced with D-Glu²⁷. In some embodiments, the stabilizing peptide has the same amino acid sequence as liraglutide except that Phe²⁸ is replaced with D-Phe²⁸. In some embodiments, the stabilizing peptide has the same amino acid sequence as liraglutide except that Ile²⁹ is replaced with D-Ile²⁹. In some embodiments, the stabilizing peptide has the same amino acid sequence as liraglutide except that Ala³⁰ is replaced with D-Ala³⁰. In some embodiments, the stabilizing peptide has the same amino acid sequence as liraglutide except that Trp³¹ is replaced with D-Trp³¹. In some embodiments, the stabilizing peptide has the same amino acid sequence as liraglutide except that Leu³² is replaced with D-Leu³². In some embodiments, the stabilizing peptide has the same amino acid sequence as liraglutide except that Val³³ is replaced with D-Val³³. In some embodiments, the stabilizing peptide has the same amino acid sequence as liraglutide except that Arg³⁴ is replaced with D-Arg³⁴. In some embodiments, the stabilizing peptide has the same amino acid sequence as liraglutide except that Arg³⁶ is replaced with D-Arg³⁶. In some embodiments, the stabilizing peptide has the same amino acid sequence as liraglutide except that the Glu attached to the side chain of Lys²⁶ is replaced with D-Glu.

In some embodiments, the stabilizing peptide has the same amino acid sequence as liraglutide except that there are two substitutions of the natural L-amino acids for D-amino acids where the two or more amino acid substitutions are sequential in the liraglutide sequence. In some embodiments, the stabilizing peptide has the same amino acid sequence as liraglutide except that there are two substitutions of an L-amino acid for a D-amino acid where the two or more amino acid substitutions are not sequential in the liraglutide sequence.

In some embodiments the glucagon-like peptide is GLP-2, a GLP-2 analogue, a derivative of GLP-2 or a derivative of a GLP-2 analogue, and the stabilizing peptide comprises GLP-2, a GLP-2 analogue, a derivative of GLP-2 or a derivative of a GLP-2 analogue that comprises at least one sequence alteration. In some embodiments, the sequence alteration is a truncation. In some embodiments, the sequence alteration is a truncation from the C-terminus. In some embodiments, the sequence alteration is a truncation from the N-terminus. In some embodiments the truncation comprises from one to ten amino acids from the C-terminus. In some embodiments, the truncation comprises from one to ten amino acids from the N-terminus.

In some embodiments, the stabilizing peptide has the same amino acid sequence as the glucagon-like peptide except that the stabilizing peptide has one or more of the amino acids in the original sequence replaced by a D-amino acid having a different sidechain than that in the original sequence. In some embodiments, the peptide has the same amino acid sequence as the glucagon-like peptide except that the stabilizing peptide has only one of the amino acids in the original sequence replaced by a D-amino acid having a different sidechain than that in the original sequence.

In some embodiments the glucagon-like peptide is GLP-2, a GLP-2 analogue, a derivative of GLP-2 or a derivative of a GLP-2 analogue, and the stabilizing peptide comprises GLP-2, a GLP-2 analogue, a derivative of GLP-2 or a derivative of a GLP-2 analogue where one or more of the L-amino acids has been replaced with a D-amino acid having the same side chain. In some embodiments, only one L-amino acid is replaced with a D-amino acid.

In some embodiments the glucagon-like peptide is exendin-4, an exendin-4 analogue, a derivative of exendin-4, or a derivative of an exendin-4 analogue, and the stabilizing peptide comprises exendin-4, an exendin-4 analogue, a derivative of exendin-4, or a derivative of an exendin-4 analogue where one or more of the L-amino acids has been replaced with a D-amino acid having the same side chain. In some embodiments, only one L-amino acid replaced with a D-amino acid.

In some embodiments, the stabilizing peptide has the same amino acid sequence as teduglutide except that the sequence alteration is a truncation of one amino acid from the C-terminus. In some embodiments, the stabilizing peptide has the same amino acid sequence as teduglutide except that the sequence alteration is a truncation of two amino acids from the C-terminus. In some embodiments, the stabilizing peptide has the same amino acid sequence as teduglutide except that the sequence alteration is a truncation of three amino acids from the C-terminus. In some embodiments, the stabilizing peptide has the same amino acid sequence as teduglutide except that the sequence alteration is a truncation of four amino acids from the C-terminus. In some embodiments, the stabilizing peptide has the same amino acid sequence as teduglutide except that the sequence alteration is a truncation of five amino acids from the C-terminus. In some embodiments, the stabilizing peptide has the same amino acid sequence as teduglutide except that the sequence alteration is a truncation of six amino acids from the C-terminus.

In some embodiments, the stabilizing peptide has the same amino acid sequence as teduglutide except that the sequence alteration is a truncation of one amino acid from the N-terminus. In some embodiments, the stabilizing peptide has the same amino acid sequence as teduglutide except that the sequence alteration is a truncation of two amino acids from the N-terminus. In some embodiments, the stabilizing peptide has the same amino acid sequence as teduglutide except that the sequence alteration is a truncation of three amino acids from the N-terminus. In some embodiments, the stabilizing peptide has the same amino acid sequence as teduglutide except that the sequence alteration is a truncation of four amino acids from the N-terminus. In some embodiments, the stabilizing peptide has the same amino acid sequence as teduglutide except that the sequence alteration is a truncation of five amino acids from the N-terminus. In some embodiments, the stabilizing peptide has the same amino acid sequence as teduglutide except that the sequence alteration is a truncation of six amino acids from the N-terminus.

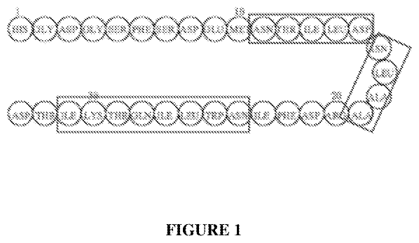

Without being bound by a specific theory, FIG. 1 highlights several regions of teduglutide thought to be involved in fibril formation. Thus, stabilizing, modifying or incorporating these specific regions in a stabilizing peptide may reduce the rate of fibril formation thereby increasing stability of the overall pharmaceutical composition. Alternatively, inclusion of these regions in the stabilizing peptide may allow for them to be sacrificed to fibril formation while the therapeutic glucagon-like peptide remains stable. Thus, in some embodiments, the stabilizing peptide is a truncation from both the C-terminus and N-terminus of the parent glucagon-like peptide such that the regions postulated to be involved in fibril formation remain. For example, in teduglutide, the stabilizing peptide may comprise amino acids 11 to 15, 16 to 19, and/or 24 to 31 of teduglutide. In some embodiments the stabilizing peptide comprises two of these fragments. In some embodiments the stabilizing peptide comprises three of these fragments. In embodiments that comprise more than one of these fragments, they may be linked via peptides that are the same or different than those in the parent glucagon-like peptide. In embodiments that comprise more than one of these fragments, they may be linked directly together.

In some embodiments, the stabilizing peptide has the same amino acid sequence as the glucagon-like peptide except that the sequence alteration in the stabilizing peptide comprises one or more amino acids in the original sequence being replaced by a D-amino acid having the same or different sidechain than that in the original sequence. In some embodiments, the stabilizing peptide has the same amino acid sequence as the glucagon-like peptide except that the stabilizing peptide has only one of the amino acids in the original sequence replaced by a D-amino acid having a different sidechain than that in the original sequence. In some embodiments, the stabilizing peptide has the same amino acid sequence as the glucagon-like peptide except that the stabilizing peptide has only one of the amino acids in the original sequence replaced by a D-amino acid having the same sidechain as that in the original sequence.

In some embodiments, the stabilizing peptide has the same amino acid sequence as the glucagon-like peptide except that the sequence alteration comprises at least two substitutions of a natural L-amino acid for a D-amino acid. The D-amino acid may have the same sidechain as the original L-amino acid or it may have a different sidechain. If the sidechain of the D-amino acid is different from that of the original L-amino acid, said sidechain is not limited to only the naturally occurring amino acids. The two or more substitutions may be sequential in the peptide sequence or they may be separated by one or more peptides in the sequence.

In some embodiments, the stabilizing peptide has the same amino acid sequence as teduglutide except that it comprises a truncation from the N-terminus and/or a truncation from the C-terminus.

The concentration of the stabilizing peptide is based on the level of stabilization provided. In some embodiments the concentration of the stabilizing peptide is a non-zero amount up to 5% w/w relative to the glucagon-like peptide. In some embodiments the concentration of the stabilizing peptide is a non-zero amount up to 4% w/w relative to the glucagon-like peptide. In some embodiments the concentration of the stabilizing peptide is a non-zero amount up to 3% w/w relative to the glucagon-like peptide. In some embodiments the concentration of the stabilizing peptide is a non-zero amount up to 2% w/w relative to the glucagon-like peptide. In some embodiments, the concentration of the stabilizing peptide is from 0.01 to 5.0% w/w relative to the glucagon-like peptide. In some embodiments, the concentration of the stabilizing peptide is from 0.25 to 4.5% w/w relative to the glucagon-like peptide. In some embodiments, the concentration of the stabilizing peptide is from 0.5 to 4.0% w/w relative to the glucagon-like peptide. In some embodiments, the concentration of the stabilizing peptide relative to the glucagon-like peptide is about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.2% w/w, about 2.4% w/w, about 2.6% w/w, about 2.8% w/w, about 3.0% w/w, about 3.2% w/w, about 3.4% w/w, about 3.6% w/w, about 3.8% w/w, about 4.0% w/w, about 4.2% w/w, about 4.4% w/w, about 4.6% w/w, about 4.8% w/w, about 5.0% w/w.

In some embodiments the composition comprises one stabilizing peptide. In some embodiments the composition comprises at least two stabilizing peptides. In some embodiments the composition comprises two stabilizing peptides. In embodiments having two or more stabilizing peptides, the sequence alteration will be different for each one. The combined amount of the two or more stabilizing peptides, relative to the glucagon-like peptide is about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.2% w/w, about 2.4% w/w, about 2.6% w/w, about 2.8% w/w, about 3.0% w/w, about 3.2% w/w, about 3.4% w/w, about 3.6% w/w, about 3.8% w/w, about 4.0% w/w, about 4.2% w/w, about 4.4% w/w, about 4.6% w/w, about 4.8% w/w, about 5.0% w/w.

The glucagon-like peptide can be produced by any method known in art. Examples include, but are not limited to, peptide synthesis, e.g. solid phase peptide synthesis using t-Boc or FMOC chemistry or other techniques known in the art. The glucagon-like peptide can also be produced by a method which comprises culturing a host cell containing a DNA sequence encoding said peptide and capable of expressing said peptide in a suitable nutrient medium under conditions permitting the expression thereof, after which it is isolated from the culture.

In some embodiments, the stabilizing peptide, because it contains at least one D-amino acid, is produced by peptide synthesis, e.g. solid phase peptide synthesis using t-Boc or Fmoc chemistry or other well established techniques. In some embodiments, the stabilizing peptide, is prepared using all L-amino acids and then subjected to conditions such that one or more of the L-amino acids racemizes to form the stabilizing peptide having one or more D-amino acids.

In some embodiments, during peptide synthesis of the stabilizing peptide, one or more of the amino acids is added to the growing peptide chain as the D-amino acid having the same side chain as the L-amino acid specific for that position in the peptide chain. In some embodiments, the stabilizing peptide is combined with the glucagon-like peptide. In some embodiments the glucagon-like peptide and the stabilizing peptide are prepare simultaneously in the same solid phase synthetic process.

In some embodiments, during peptide synthesis of the stabilizing peptide, if the sequence alteration is a truncation on the N-terminus, a small percentage of the growing peptide chain can be endcapped to prevent further elongation while the remaining portion of the chain is extended. Alternatively, if the sequence alteration is a truncation on the C-terminus, a small amount of new resin can be added to the growing sequence after several amino acids have already been coupled. In some embodiments, the stabilizing peptide is combined with the glucagon-like peptide. For either option, the final isolated composition comprises both the glucagon-like peptide and the stabilizing peptide.

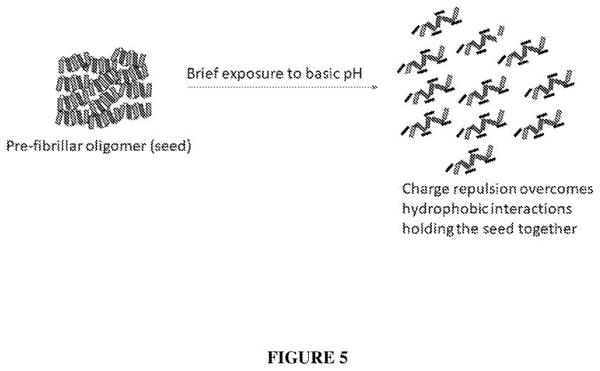

Without being bound by a specific theory, as shown in FIG. 5, it is thought that fibril formation begins with the initial formation of “seeds” that serve as anchor points for the unfolding and refolding of the glucagon-like protein into fibrils in a manner analogous to nucleation during the recrystallization of small molecules. Disruption of seed formation and/or reduction of seeds present in the glucagon-like protein may reduce the rate in which fibrils form thereby stabilizing the composition. It may also increase the lag-time before fibrils begin to form. Thus, treating the glucagon-like protein with base in order to disrupt seed formation and/or reduce the number of seeds present should prolong the stability of the glucagon-like protein.

Thus, in some embodiments, the glucagon-like protein is treated with base before being incorporated into a pharmaceutical composition. Treating the glucagon-like protein with base comprises stirring, or otherwise agitating, an aqueous solution of the glucagon-like protein and base for a predetermined period of time. In some embodiments, treating further comprises lyophilizing the aqueous solution after stirring the glucagon-like protein with base thereby creating the stabilized glucagon-like protein. This stabilized glucagon-like protein can be used in any of the methods and compositions described elsewhere herein. In some embodiments, the stabilizing peptide is added to the glucagon-like protein and stirred with the base. In some embodiments, the glucagon-like protein and stabilizing peptide are lyophilized thereby creating a stabilized protein and peptide mixture. This mixture can be used in any of the methods and compositions described elsewhere herein.

In some embodiments, the base is selected from the group consisting of hydroxides, carbonates, bicarbonates, phosphates, amines and combinations thereof. For inorganic bases, the counterion can be any pharmaceutically acceptable counterion. For example, in some embodiments, the counterion is selected from the group consisting of ammonium, sodium, potassium, calcium, magnesium, and lithium. More specifically, in some embodiments, the base is ammonium hydroxide. In some embodiments, the base is sodium hydroxide. In some embodiments, the base is potassium hydroxide. In some embodiments, the base is calcium hydroxide. In some embodiments, the base is magnesium hydroxide. In some embodiments, the base is sodium carbonate. In some embodiments, the base is potassium carbonate. In some embodiments, the base is ammonium carbonate. In some embodiments, the base is magnesium carbonate. In some embodiments, the base is sodium phosphate. In some embodiments, the base is potassium phosphate. In some embodiments, the base is ammonium phosphate. In some embodiments, the base is magnesium phosphate.

In some embodiments, the glucagon-like protein is treated with base before being incorporated into the pharmaceutical composition using any pharmaceutically acceptable base where the pH is adjusted to greater than 7.0. In some embodiments, the pH is between 7.1 and 13.0. In some embodiments, the pH is between 7.5 and 12.5. In some embodiments, the pH is between 8.0 and 12.0. In some embodiments, the pH is adjusted to about 7.5, 8.0, 8.5, 9.0, 9.5, 10.0, 10.5, 11.0, 11.5, 12.0, 12.5, or 13.0.

In some embodiments, the concentration of the base is from about 0.01 M to about 5.0 M. In some embodiments, the concentration of the base is from about 0.02 M to 4.0 M. In some embodiments, the concentration of the base is from about 0.03 M to 3.0 M. In some embodiments, the concentration of the base is from about 0.05 M to 2.0 M. In some embodiments, the concentration of the base is from about 0.1 M to 1.5 M. In some embodiments, the concentration of the base is from about 0.1 M to 1.0 M. In some embodiments, the concentration of the base is about 0.01 M, about 0.05 M, about 0.1 M, about 0.5 M, about 1.0 M, or about 2.0 M.

In some embodiments, treating the glucagon-like protein with base comprises stirring said protein in an aqueous solution of the base for from 10 minutes to 6 hours. In some embodiments, the treatment is for from 30 minutes to 4 hours. In some embodiments, the treatment is for from 45 minutes to 3 hours. In some embodiments, the treatment is for from 60 minutes to 2 hours. In some embodiments, the treatment is for 30 minutes. In some embodiments, the treatment is for 60 minutes. In some embodiments, the treatment is for 90 minutes. In some embodiments, the treatment is for 2 hours. In some embodiments, the treatment is for 2.5 hours. In some embodiments, the treatment is for 3 hours. In some embodiments, the treatment is for 5 hours.

In some embodiments, during treatment with base, the glucagon-like protein has a concentration of from 1 mg/mL to 25 mg/mL. In some embodiments, during treatment with base, the glucagon-like protein has a concentration of from 2 mg/mL to 20 mg/mL. In some embodiments, during treatment with base, the glucagon-like protein has a concentration of from 3 mg/mL to 15 mg/mL. In some embodiments, during treatment with base, the glucagon-like protein has a concentration of from 4 mg/mL to 10 mg/mL. In some embodiments, during treatment with base, the glucagon-like protein has a concentration of from 5 mg/mL to 10 mg/mL. In some embodiments, the concentration of the glucagon-like protein during the treatment with base is about 1 mg/mL, about 2 mg/mL, 3 mg/mL, about 4 mg/mL, 5 mg/mL, about 6 mg/mL, 7 mg/mL, about 8 mg/mL, 9 mg/mL, about 10 mg/mL, about 15 mg/mL, about 20 mg/mL, or about 25 mg/mL.

The lag-time, as measured according to the ThT assay in the Examples, is the time period before which fibrils begin to form. A composition having a longer lag-time is generally stable for a longer time period compared to a composition with a shorter lag-time. Thus, an increase in the lag-time for fibril formation is beneficial for the composition. In some embodiments, the pharmaceutical composition prepared according to the methods described herein has a longer lag-time as compared to an identical pharmaceutical composition except that no stabilizing peptide or stabilizing treatment was used for preparation of the composition. In some embodiments, the lag-time is at least 25% greater. In some embodiments, the lag-time is at least 50% greater. In some embodiments, the lag-time is at least 75% greater. In some embodiments, the lag-time is at least 100% greater. In some embodiments, the lag-time is at least 150% greater. In some embodiments, the lag-time is at least 200% greater.

Also disclosed herein is a stabilized bulk protein composition, the composition comprises a glucagon-like protein and a stabilizing peptide, wherein the glucagon-like protein and the stabilizing peptide have been treated with base. The glucagon-like protein and a stabilizing peptide are as described elsewhere herein. Treating with base is done as described elsewhere herein. Said bulk, stabilized protein composition can be used in any of the compositions and methods disclosed elsewhere herein. In some embodiments, the glucagon-like protein is teduglutide. In some embodiments, the stabilizing peptide is a [1-30] fragment of teduglutide. In some embodiments, the base is ammonium hydroxide. In some embodiments, the glucagon-like protein is liraglutide. In some embodiments, the stabilizing peptide comprises a single amino acid substitution with its D-isomer.

The pharmaceutical compositions disclosed herein can be administered to patients in need of treatment therewith using any appropriate route. For example, the composition can be administered orally or parenterally to said patients. Types of parenteral administration include, but are not limited to, injection, infusion, and implantation. Typical types of injection or infusion include, but are not limited to, intravenous, intraosseous, intramuscular, subcutaneous, epidural, and intradermal. In some embodiments, the composition is administered subcutaneously. In some embodiments, the composition is administered intravenously.

The amount of the pharmaceutical composition administered to a patient in need of treatment therewith is based on the specific needs of said patient and determined by the medical professional overseeing said treatment. Because the pharmaceutical composition can be prepared at different concentrations, the dosage of the composition administered to the patient in need thereof is based on the amount of the glucagon-like peptide. In some embodiments the patients receives a dosage of the pharmaceutical composition such that said patient receives from about 0.1 to about 15 mg of the glucagon-like peptide. In some embodiments the patients receives a dosage of the pharmaceutical composition such that said patient receives from about 0.1 to about 15 mg of the glucagon-like peptide. In some embodiments the patients receives a dosage of the pharmaceutical composition such that said patient receives from about 0.6 to about 12 mg of the glucagon-like peptide. In some embodiments the patients receives a dosage of the pharmaceutical composition such that said patient receives about 0.6 mg of the glucagon-like peptide. In some embodiments the patients receives a dosage of the pharmaceutical composition such that said patient receives about 1.2 mg of the glucagon-like peptide. In some embodiments the patients receives a dosage of the pharmaceutical composition such that said patient receives about 1.8 mg of the glucagon-like peptide. In some embodiments the patients receives a dosage of the pharmaceutical composition such that said patient receives about 2.4 mg of the glucagon-like peptide. In some embodiments the patients receives a dosage of the pharmaceutical composition such that said patient receives about 3.0 mg of the glucagon-like peptide. In some embodiments the patients receives a dosage of the pharmaceutical composition such that said patient receives about 3.5 mg of the glucagon-like peptide. In some embodiments the patients receives a dosage of the pharmaceutical composition such that said patient receives about 4.0 mg of the glucagon-like peptide. In some embodiments the patients receives a dosage of the pharmaceutical composition such that said patient receives about 4.5 mg of the glucagon-like peptide. In some embodiments the patients receives a dosage of the pharmaceutical composition such that said patient receives about 5.0 mg of the glucagon-like peptide. In some embodiments the patients receives a dosage of the pharmaceutical composition such that said patient receives about 5.5 mg of the glucagon-like peptide. In some embodiments the patients receives a dosage of the pharmaceutical composition such that said patient receives about 6.0 mg of the glucagon-like peptide. In some embodiments the patients receives a therapeutically effective amount of the pharmaceutical composition.

The patient may be administered the pharmaceutical composition by a medical professional, or the patient (or said patient's caregiver) may administer the pharmaceutical composition.

For any of the methods disclosed herein, the pharmaceutical composition is as described elsewhere herein. Any composition disclosed herein can be used with any method herein.

Solid phase peptide synthesis is known in the art. An amino acid is attached to a solid phase particle by a linking group on the acid side with a protecting group on the amine side. The protecting group is removed so that the second amino acid (via its acid group) can be coupled to the amine group on the original acid. The second (and succeeding) acids are also initially protected, and thus the general sequence is to deprotect, couple, and repeat until the desired peptide is completed, following which the completed peptide is cleaved from the solid phase resin.

Also disclosed herein is a method for preparing an aqueous stabilized peptide composition, the method comprises dissolving the buffer in water to make Solution A; dissolving the stabilizing peptide in Solution A to make Solution B; dissolving the glucagon-like peptide in Solution B to make Solution C; dissolving the isotonicity agent in water to make Solution D; combining Solution C and Solution D to make the Combined Solution; adjusting the pH of the Combined Solution to between 6.0 and 9.0 using either a pharmaceutically acceptable aqueous acid or pharmaceutically acceptable aqueous base as needed; and optionally adding water to the Combined Solution. The solutions are mixed when being combined to ensure homogeneous and efficient combination of the ingredients.

In preparation of the Combined Solution, the order in which the ingredients are combined may vary. For example, the glucagon-like peptide may be dissolved in the buffer prior to or after the stabilizing peptide is added.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In some instances, the Combined Solution is lyophilized to form a dry solid that can be stored for at least several months. In some embodiments, the lyophilized solid is stable to storage for up to 30 months. In some embodiments, it is then reconstituted by the addition of water prior to dispensing, packaging, and/or administering to a patient in need thereof.

In one non-limiting example, the filtered solution or the reconstituted solution is dispensed into pre-filled syringes. In yet another non-limiting example, the filtered solution or the reconstituted solution is dispensed into standard sterile vials with a septum cap. In yet another non-limiting example, the filtered solution or the reconstituted solution is dispensed into a cartridge assembly for a pen device. In yet another non-limiting example, the dry lyophilized solid is packaged in a vial that is reconstituted by the addition of a predetermined amount of sterile water just prior to administration to a patient.

Also disclosed herein is a method for preparing a stabilized peptide composition. The composition comprises a glucagon-like peptide and a stabilizing peptide. In some embodiments, the glucagon-like peptide and a stabilizing peptide have the same amino acid sequence except that the stabilizing peptide comprises a sequence alteration that is a truncation.

In some embodiments, the stabilized peptide composition comprising the glucagon-like peptide and a stabilizing peptide are made using solid phase peptide synthesis with the glucagon-like peptide and the stabilizing peptide being prepared simultaneously as a mixture in the same process. For example, if the sequence alteration in the stabilizing peptide is a truncation on the N-terminus of the peptide chain, after the nitrogen is deprotected in preparation for the next coupling step, from 0.1 to 5.0% of the unprotected can be endcapped (e.g. acetylation) to prevent additional coupling which stops elongation of the chain. The coupling and chain elongation then resume with the remaining uncapped peptide chain.

In some embodiments, when the glucagon-like peptide and a stabilizing peptide are prepared as a mixture, disclosed herein is a method for preparing an aqueous stabilized peptide composition, the method comprises dissolving the buffer in water to make Solution A; dissolving the stabilizing peptide and glucagon-like peptide mixture in Solution A to make Solution C; dissolving the isotonicity agent in water to make Solution D; combining Solution C and Solution D while mixing to make the Combined Solution; adjusting the pH of the Combined Solution to between 6.0 and 9.0 using either a pharmaceutically acceptable aqueous acid or pharmaceutically acceptable aqueous base as needed; and optionally adding water to the Combined Solution.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In one non-limiting example, the filtered solution is dispensed into pre-filled syringes. In yet another non-limiting example, the filtered solution is dispensed into standard sterile vials with a septum cap. In another non-limiting example, the filtered solution is dispensed into a cartridge assembly for a pen device.

Also disclosed herein is a method for making a stabilized peptide composition comprising glucagon-like peptide and a stabilizing peptide. In some embodiments, both the glucagon-like peptide and the stabilizing peptide are prepared separately using traditional solid phase techniques. After both are completed and individually purified, the stabilizing peptide is added to the glucagon-like peptide. The combined peptides are then dried. In some aspects, they are freeze dried. The ratio of the glucagon-like peptide to the stabilizing peptide is as described elsewhere herein. The stabilizing peptide is as described elsewhere herein.

In yet another aspect, disclosed herein is a method for preparing an aqueous stabilized peptide composition, the method comprises dissolving the buffer, isotonicity agent and preservative in water to make Solution A; dissolving the glucagon-like peptide in water to make Solution B; dissolving the stabilizing peptide in water to make Solution C; combining Solution A, Solution B and Solution C while mixing to make the Combined Solution; adjusting the pH of the Combined Solution to between 6.0 to 12.0 using either a pharmaceutically acceptable aqueous acid or pharmaceutically acceptable aqueous base as needed; and optionally adding water to the Combined Solution.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In one non-limiting example, the filtered solution is dispensed into pre-filled syringes. In yet another non-limiting example, the filtered solution is dispensed into standard sterile vials with a septum cap. In yet another non-limiting example, the filtered solution is dispensed into a cartridge assembly for a pen device.

In some embodiments, disclosed herein is a method for preparing an aqueous stabilized peptide composition, the method comprises dissolving the buffer, isotonicity agent and preservative in water to make Solution A; dissolving the glucagon-like peptide in water to make Solution B; dissolving the stabilizing peptide in water to make Solution C; combining Solution A and Solution B while mixing to make an Intermediate Solution; adjusting the pH of the Intermediate Solution to between 6.0 to 12.0 using either a pharmaceutically acceptable aqueous acid or pharmaceutically acceptable aqueous base as needed; adding Solution C to the Intermediate Solution to make a Combined Solution; and optionally adding water to the Combined Solution. In some embodiments the pH is adjusted to between 6.0 and 12.0 before Solution C is added to the Intermediate Solution. In some embodiments the pH is adjusted to between 6.0 and 12.0 after Solution C is added to the Intermediate Solution.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In one non-limiting example, the filtered solution is dispensed into pre-filled syringes. In yet another non-limiting example, the filtered solution is dispensed into standard sterile vials with a septum cap. In yet another non-limiting example, the filtered solution is dispensed into a cartridge assembly for a pen device.

In some embodiment, disclosed herein is a method for preparing an aqueous stabilized peptide composition, the method comprises dissolving the buffer, the isotonicity agent and preservative in water to make Solution A; adding the active agent to Solution A to make an Intermediate Solution; adjusting the pH of the Intermediate Solution to between 6.0 to 12.0 using either a pharmaceutically acceptable aqueous acid or pharmaceutically acceptable aqueous base as needed; adding the stabilizing peptide to the Intermediate Solution to make the Combined Solution; and optionally adding water to the Combined Solution. In some embodiments, the pH of the solution is adjusted to between 6.0 and 12.0 before the stabilizing peptide is added to the Intermediate Solution. In some embodiments, the pH of the solution is adjusted to between 6.0 and 12.0 after the stabilizing peptide is added to the Intermediate Solution.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In one non-limiting example, the filtered solution is dispensed into pre-filled syringes. In yet another non-limiting example, the filtered solution is dispensed into standard sterile vials with a septum cap. In yet another non-limiting example, the filtered solution is dispensed into a cartridge assembly for a pen device.

Also disclosed herein is a method for preparing a stabilized peptide composition. The composition comprises a glucagon-like peptide and a stabilizing peptide. In some embodiments, the glucagon-like peptide and a stabilizing peptide have the same amino acid sequence except that one or more amino acids in the stabilizing peptide is a D-amino acid rather than an L-amino acid.

In some embodiments, the stabilized peptide composition comprising the glucagon-like peptide and a stabilizing peptide are made using solid phase peptide synthesis with the glucagon-like peptide and the stabilizing peptide being prepared simultaneously as a mixture in the same process. During the solid phase synthesis, one or more the amino acids in the growing peptide is added as a mixture of the L-amino acid and the D-amino acid rather than as the pure L-amino acid. The ratio of the L-amino acid to the D-amino acid is such that the final peptide composition is stabilized in aqueous solution as compared to a solution without the stabilizing peptide. In some embodiments, the ratio of the L-amino acid to the D-amino acid is 97:3 to 99.9:0.1 w/w. In some embodiments, the ratio of the L-amino acid to the D-amino acid is 98:2 w/w. In some embodiments, the ratio of the L-amino acid to the D-amino acid is 99:1 w/w. In some embodiments, the D-amino acid comprises up to about 5 wt % of the mixture of L-amino acid to D-amino acid.

In some embodiments, when the glucagon-like peptide and a stabilizing peptide are prepared as a mixture, disclosed herein is a method for preparing an aqueous stabilized peptide composition, the method comprises dissolving the buffer, isotonicity agent and preservative in water to make Solution A; dissolving the glucagon-like peptide and the stabilizing peptide in water to make Solution B; combining Solution A and Solution B while mixing to make the Combined Solution; adjusting the pH of the Combined Solution to between 6.0 to 12.0 using either a pharmaceutically acceptable aqueous acid or pharmaceutically acceptable aqueous base as needed; and optionally adding water to the Combined Solution.

In some embodiments, the method further comprises filtering the Combined Solution. In some embodiments, the filtering step comprises using at least one sub-micron filter. In some embodiments, the filtered solution is dispensed into one or more pharmaceutically acceptable containers. In one non-limiting example, the filtered solution is dispensed into pre-filled syringes. In yet another non-limiting example, the filtered solution is dispensed into standard sterile vials with a septum cap. In another non-limiting example, the filtered solution is dispensed into a cartridge assembly for a pen device.

Also disclosed herein is a method for making a stabilized peptide composition comprising glucagon-like peptide and a stabilizing peptide. In some embodiments, both the glucagon-like peptide and the stabilizing peptide are prepared separately using traditional solid phase techniques. After both are completed and individually purified, the stabilizing peptide is added to the glucagon-like peptide. The combined peptides are then dried. In some aspects, they are freeze dried. The ratio of the glucagon-like peptide to the stabilizing peptide is as described elsewhere herein. The stabilizing peptide is as described elsewhere herein.

Also disclosed herein a method for making a stabilized peptide composition comprising glucagon-like peptide and a stabilizing peptide. In some embodiments, glucagon-like peptide is prepared using traditional solid phase techniques. After the glucagon-like peptide is cleaved from the resin, the peptide is stirred in a solution, and the pH is adjusted so that a one or more of the amino acids in the glucagon-like peptide racemizes thereby forming a mixture of the glucagon-like peptide and the stabilizing peptide. The mixture undergoes controlled purification and is then dried, preferably freeze-dried. The racemization is carried out using conditions such that the stabilizing peptide is formed as described elsewhere herein. The ratio of the glucagon-like peptide to the stabilizing peptide is as described elsewhere herein. In some embodiments, the stabilizing peptide and the glucagon-like peptide are isolated together during purification, thereby creating a mixture that comprises the glucagon-like peptide and the stabilizing peptide. In some embodiments, the stabilizing peptide and the glucagon-like peptide are separated from one another during purification with each being isolated separately from the other. In some embodiments, the purification is performed such that the concentration of the glucagon-like peptide is enhanced as compared to its concentration in the composition prior to racemization. In some embodiments, the purification is performed such that the concentration of the stabilizing peptide is enhanced as compared to its concentration in the composition prior to the racemization. In any embodiment, the glucagon-like peptide and the stabilizing peptide can be used as described in any of the methods and compositions described herein.

By way of example and not limitation, if the racemization procedure results in a ratio of the glucagon-like peptide to stabilizing peptide of 95:5, the purification is controlled in one of three possible options. In one embodiment, the purification is controlled such that the final ratio of the two peptides is still 95:5. In another embodiment, the purification is controlled such that each of the two peptides is isolated in completely pure form separate from the other. In another embodiment, the purification is controlled such that the concentration of the glucagon-like peptide relative to the stabilizing peptide increases resulting in a final ratio of 99:1. The final ratio would be controlled such that the ratio of the glucagon-like peptide to the stabilizing peptide is as described elsewhere herein. In any embodiment, the glucagon-like peptide and the stabilizing peptide can be used in any of the methods and compositions described elsewhere herein.

This written description uses examples to disclose the invention herein to enable any person skilled in the art to practice the invention, including making and using any compositions and performing any incorporated methods. Any aspect or embodiment disclosed herein may be used in combination with any other aspect or embodiment as would be understood by a person skilled in the art. Other examples are intended to be within the scope of the claims if they have structural elements that do not differ from the literal language of the claims, or if they include equivalent structural elements with insubstantial differences from the literal languages of the claims.

### Examples and Procedures

### Thioflavin T (ThT) Fibrillation Assay: Principle and Examples

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki, et al. (1989) Anal. Biochem., 177, 244-249; Le Vine (1999) Methods. Enzymol., 309, 274-284].

The time course for fibril formation can be described by a sigmoidal curve with the following expression [Nielsen, et al. (2001) Biochemistry, 40, 6036-6046]:

$$F = {f}_{i} + {m}_{i} τ + \frac{{f}_{f} + {m}_{f} t}{1 + {e}^{- [ ( t - {t}_{0} ) / τ ]}} \qquad Equation ( 1 )$$

where F is the ThT fluorescence at the time t, and t₀ is the time needed to reach 50% of maximum fluorescence. The two important parameters describing fibril formation are the lag-time calculated by t₀−2τ and the apparent rate constant kₐₚₚ=1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

### Sample Preparation for Teduglutide Compositions

Sample aliquots of 99.9 μL and 11.1 μL of a 50 μM solution of ThT in Phosphate Buffered Saline were placed in a 96 well microtiter plate. Usually, five replica of each sample (corresponding to one test condition) were placed in one column of wells. The plate was sealed with a sealing tape (Corning). Vials of Gattex and Revestive was reconstituted in 0.54 mL of sterile water. The solution thus obtained was used for the ThT assay as described above.

### Incubation and Fluorescence Measurement

Incubation of the stabilized peptide composition (teduglutide and the stabilizing peptide) in PBS in a 96-well plate was at 25° C.; without shaking and measurement of the ThT fluorescence emission were done in an Enspire 2300 plate reader (Perkin Elmer). Fluorescence measurement was done using excitation through a 440 nm filter and measurement of emission through a 482 nm filter.

The plate was measured every 60 minutes for 20 hours.

### Data Handling

The measurement points were saved in Microsoft Excel format for further processing and curve drawing. The background emission from ThT in the absence of fibrils was negligible. The data points are typically a mean of five samples. Only data obtained in the same experiment (i.e. samples on the same plate) are presented in the same graph ensuring a relative measure of fibrillation between the individual samples of one assay rather than comparison between different assays.

The degree of fibrillation is expressed as ThT fluorescence at various time points calculated as the mean of five samples.

Teduglutide Composition Stabilized with a Stabilizing Peptide

A 0.1 L batch of a stabilized teduglutide composition was prepared according to the method described herein. A 65 mL solution of teduglutide, stabilizing peptide (teduglutide fragment [1-30] (3 wt % or 4 wt % relative to teduglutide), disodium hydrogen phosphate heptahydrate, and sodium dihydrogen phosphate monohydrate in water were mixed until all reagents dissolved (Solution A). A 30 mL solution of L-histidine, Mannitol and water was mixed until all reagents dissolved (Solution B). Solutions A and B were slowly combined with mixing. The pH of the solution was adjusted to 7.4±0.1 using either HCl(aq) or NaOH(aq) as required. The final volume of the batch was adjusted to 0.1 L using additional water to obtain a concentration of disodium hydrogen phosphate heptahydrate (6.868 mg/mL), sodium dihydrogen phosphate monohydrate (1.288 mg/mL), mannitol (30 mg/mL), L-histidine (7.76 mg/mL), Teduglutide (10 mg/mL) and stabilizing peptide (0.3 or 0.4 mg/mL). The entire solution was filtered through a 0.2 μm filter. The final solution was used in a ThT assay.

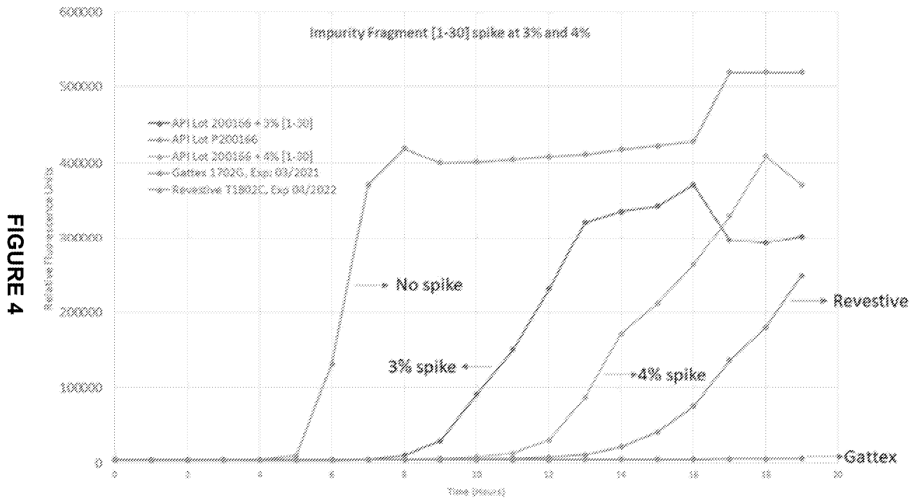

In FIG. 4, a stabilizing peptide with a three amino acid truncation on the N-terminus that otherwise has the same amino acid sequence as teduglutide was prepared and tested. Gattex®, Revestive®, and teduglutide without a stabilizing peptide are used for comparison and controls.

### Based Stabilized Teduglutide

Prior to incorporation into a pharmaceutical composition, the teduglutide was stabilized with base treatment according to the following procedure. The protein was dissolved in 0.1 M ammonium hydroxide and allowed to stand for a either one or two hours. A stabilizing peptide, fragment [1-30], was then added at a concentration of 3.5 w/w %. The mixture was then lyophilized. The lyophilized composition was then incorporated into a pharmaceutical composition as described in the previous example.

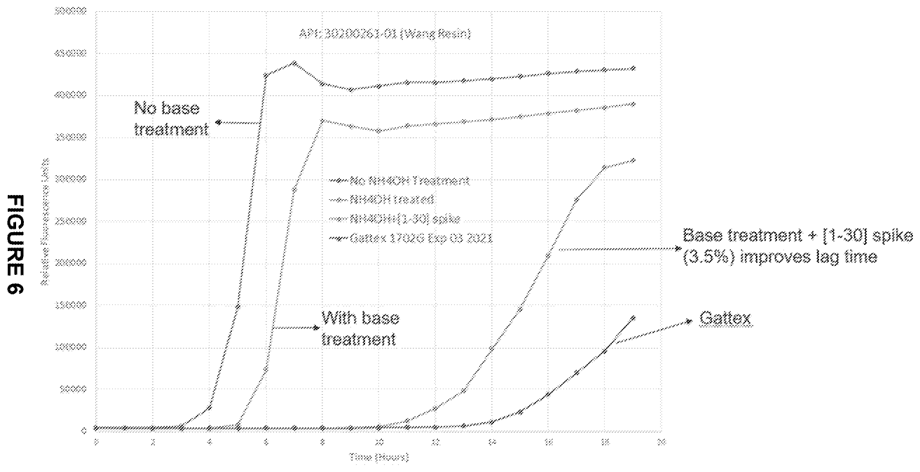

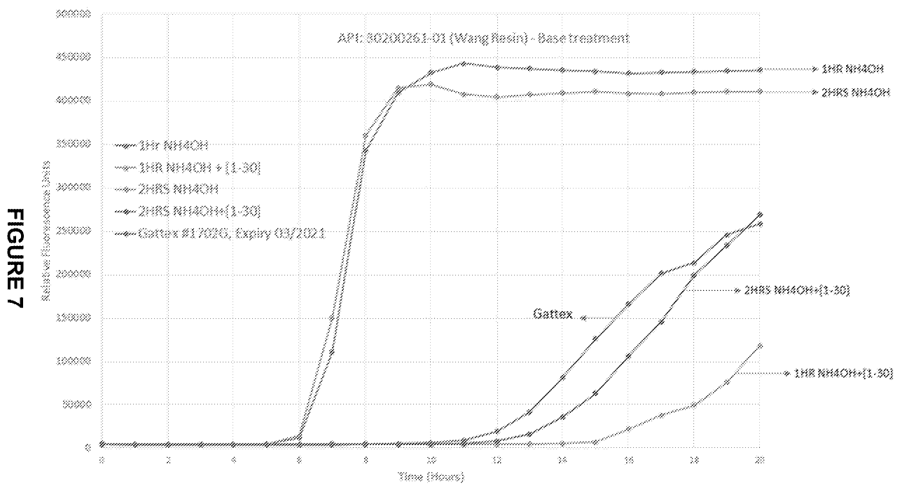

In FIGS. 6 and 7, the results of the ThT assay are shown comparing three different formulations: base+fragment [1-30] stabilized, fragment [1-30] stabilized, and unstabilized. Both samples treated with base for either 1 hour (FIG. 6) or 2 hours (FIG. 7) were more stable to fibril formation exhibiting a significantly increased lag time as compared to control samples.

### Sample Preparation for Liraglutide Compositions

Sample aliquots of 200 μL and 22.2 μL of a 50 μM solution of ThT in Phosphate Buffered Saline were placed in a 96 well microtiter plate. Usually, six replica of each sample (corresponding to one test condition) were placed in one column of wells. The plate was sealed with a sealing tape (Corning).

### Incubation and Fluorescence Measurement

Incubation of the stabilized peptide composition (liraglutide and 1 wt % of the stabilizing peptide) in PBS in a 96-well plate was at 37° C.; shaking and measurement of the ThT fluorescence emission were done in an Enspire 2300 plate reader (Perkin Elmer). The orbital shaking was adjusted to 600 rpm with an amplitude of 1 mm in all the presented data. Fluorescence measurement was done using excitation through a 440 nm filter and measurement of emission through a 482 nm filter.

The plate was measured every 60 minutes for 95 hours. Between each measurement, the plate was shaken and heated as described.

### Data Handling

The measurement points were saved in Microsoft Excel format for further processing and curve drawing. The background emission from ThT in the absence of fibrils was negligible. The data points are typically a mean of six samples. Only data obtained in the same experiment (i.e. samples on the same plate) are presented in the same graph ensuring a relative measure of fibrillation between the individual samples of one assay rather than comparison between different assays.

The degree of fibrillation is expressed as ThT fluorescence at various time points calculated as the mean of six samples.

### Stabilized Liraglutide Composition

A 0.1 L batch of a stabilized liraglutide composition was prepared. A 60 mL solution of sodium dihydrogen phosphate, propylene glycol and phenol in water was mixed until all reagents dissolved (Solution A). A 30 mL solution of liraglutide and water was mixed until it dissolved (Solution B). A 3 mL solution of a stabilizing peptide (liraglutide amino acid sequence with Ser¹⁴ to D-Ser¹⁴ substitution) (1 wt % relative to liraglutide) in water was mixed until it dissolved (Solution C). Solutions A, B and C were slowly combined with mixing. The pH of the solution was adjusted to 8.1-8.2 using either HCl(aq) or NaOH(aq) as required. The final volume of the batch was adjusted to 0.1 L using additional water to obtain a concentration of sodium dihydrogen phosphate (1.42 mg/mL), propylene glycol (14 mg/mL), phenol (5.5 mg/mL), liraglutide (6 mg/mL) and stabilizing peptide (0.06 mg/mL). The entire solution was filtered through a 0.5 μm filter followed by a 0.2 μm filter. The final solution was then packaged or used in a ThT assay.

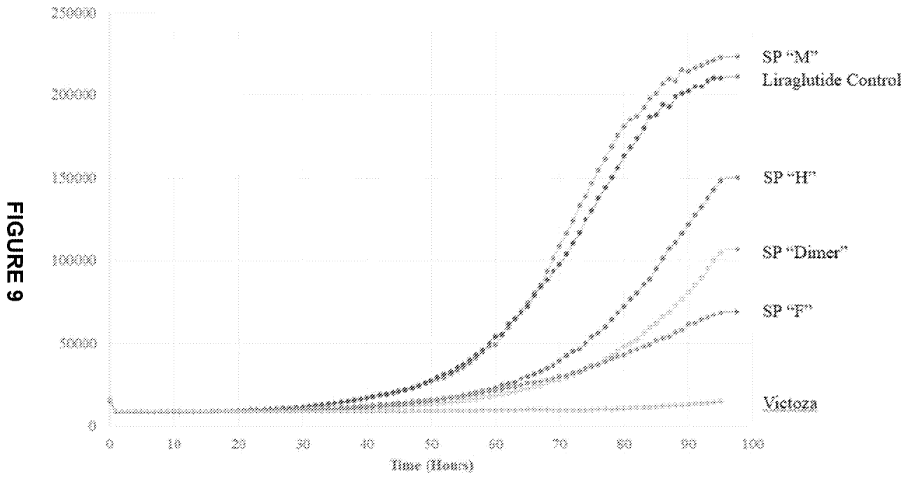

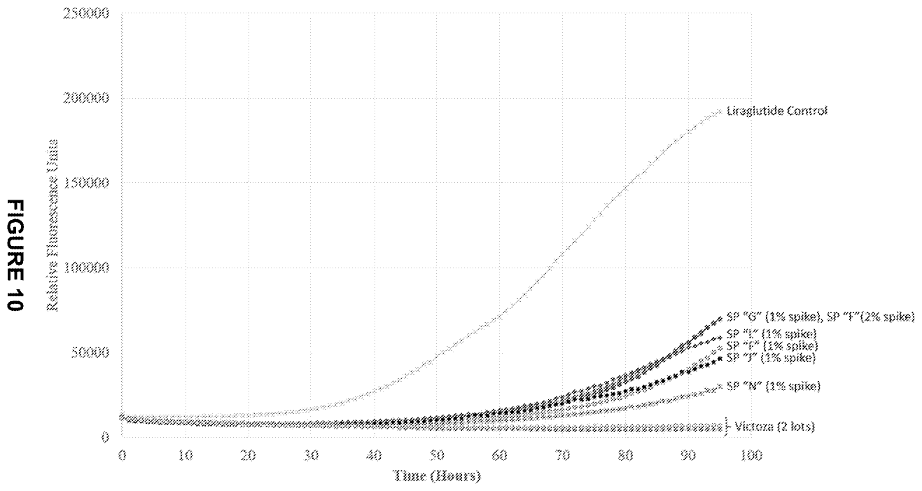

Using this same procedure, multiple batches, each with a different stabilizing peptide, were prepared and their stability was tested via the ThT assay. FIGS. 9 and 10 illustrate the results from several different stabilizing peptides. Each stabilizing peptide has the same amino acid sequence as liraglutide (see FIG. 3 for the liraglutide amino acid sequence) except for the single substitution of a D-amino acid for an L-amino acid. The table below illustrates the amino acid position substituted along with its weight percentage relative to the liraglutide. “Dimer” is a liraglutide dimer prepared by exposure of liraglutide to UV for 22 hours. Victoza and liraglutide without a stabilizing peptide are used for comparison and controls.

| Seq. Position | ID | Amino Acid | wt % |
|---|---|---|---|
| 7 | SP M | His | 1% |
| 13 | SP L | Thr | 1% |
| 14 | SP F | Ser | 1%/2% |
| 15 | SP N | Asp | 1% |
| 17 | SP G | Ser | 1% |
| 18 | SP H | Ser | 1% |
| 30 | SP J | Ala | 1% |
| N/A | Dimer | N/A | 1% |

1. An aqueous stabilized peptide composition, said composition comprising a glucagon-like peptide, and a stabilizer.

2. The aqueous stabilized peptide composition according to claim 1, wherein the glucagon-like peptide is selected from group consisting of glucagon, a glucagon analogue, a glucagon derivative, oxynthomodulin, GLP-1, a GLP-1 analogue, a derivative of GLP-1, a derivative of a GLP-1 analogue, GLP-2, a GLP-2 analogue, a derivative of GLP-2, a derivative of a GLP-2 analogue, exendin-4, an exendin-4 analogue, a derivative of exendin-4, and a derivative of an exendin-4 analogue.

3. The aqueous stabilized peptide composition according to claim 1, wherein the glucagon-like peptide is selected from the group consisting of GLP-2 a GLP-2 analogue, a derivative of GLP-2, and a derivative of a GLP-2 analogue.

4. The aqueous stabilized peptide composition according to claim 3, wherein the glucagon-like peptide is teduglutide.

5. The aqueous stabilized peptide composition according to claim 1, wherein the glucagon-like peptide is selected from the group consisting of GLP-1, a GLP-1 analogue, a derivative of GLP-1, and a derivative of a GLP-1 analogue.

6. The aqueous stabilized peptide composition according to claim 5, wherein the glucagon-like peptide is liraglutide.

7. The aqueous stabilized peptide composition according to claim 1, wherein the aqueous stabilized peptide composition further comprises a buffer selected from the group consisting of phosphate, TRIS, PBS, glycine, N-glycylglycine, sodium acetate, sodium carbonate, glycylglycine, histidine, lysine, arginine, sodium citrate and combinations thereof.

8. The aqueous stabilized peptide composition according to claim 7, wherein the buffer comprises phosphate.

9. The aqueous stabilized peptide composition according to claim 8, wherein the phosphate buffer has a pH in a range of from 6.0 to 12.0.

10. The aqueous stabilized peptide composition according to claim 1, wherein the aqueous stabilized peptide composition further comprises an isotonicity agent selected from the group consisting of sodium chloride, xylitol, mannitol, sorbitol, glycerol, glucose, maltose, sucrose, L-glycine, L-histidine, arginine, lysine, isoleucine, aspartic acid, tryptophan, threonine, dimethyl sulfone, polyethylene glycol, propylene glycol and combinations thereof.

11. The aqueous stabilized peptide composition according to claim 10, wherein the isotonicity agent is mannitol or propylene glycol.

12. The aqueous stabilized peptide composition according to claim 1, wherein the aqueous stabilized peptide composition further comprises a stabilizer selected from the group consisting of polyvinyl pyrrolidone, polyvinyl alcohol-polyethylene glycol, Pluronic F68, tocopherol polyethylene glycol succinate, hydroxypropyl methylcellulose, hydroxyethyl cellulose, hydroxypropyl cellulose, methylcellulose, carboxymethylcellulose sodium, polyvinyl alcohol, sodium alginate, Tween 80, an amino acid (e.g., L-histidine), and a stabilizing peptide, surfactants, xanthan gum, acacia, and combinations thereof.

13. The aqueous stabilized peptide composition according to claim 12, wherein the stabilizer is L-histidine.

14. The aqueous stabilized peptide composition according to claim 12, wherein the stabilizer is a stabilizing peptide, wherein said stabilizing peptide has the same amino acid sequence as the glucagon-like peptide wherein the stabilizing peptide comprises at least one sequence alternation when compared to the glucagon-like peptide.

15. The aqueous stabilized peptide composition according to claim 1, wherein the aqueous stabilized peptide composition further comprises a preservative selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thimerosal, and combinations thereof.

16. The aqueous stabilized peptide composition according to claim 15, wherein the preservative is phenol.

17. An aqueous stabilized peptide composition, said composition comprising
10 mg/mL of teduglutide,
6.87 mg/mL of dibasic sodium phosphate heptahydrate,
1.29 mg/mL of monobasic sodium phosphate monohydrate,
7.76 mg/mL of L-histidine,
30 mg/mL of mannitol, and
4% w/w, relative to teduglutide, a stabilizing peptide that has the same amino acid sequence as teduglutide wherein said stabilizing peptide has a three amino acid truncation on the C-terminus.

18. A method for making the stabilized peptide composition according to claim 17, said method comprising: a) dissolving the dibasic sodium phosphate heptahydrate and monobasic sodium phosphate monohydrate in water to make Solution A; b) adding the stabilizing peptide to Solution A to make Solution B; c) adding the teduglutide to Solution B to make Solution C; d) dissolving the L-histidine and mannitol in water to make Solution D; e) combining Solution D and Solution C to make the Combined Solution; and f) adjusting the pH of the Combined Solution to between 6.0 and 9.0 using either HCl(aq) or NaOH(aq).

19. An aqueous stabilized peptide composition, said composition comprising
6 mg/mL of liraglutide,
1.42 mg/mL of sodium hydrogen phosphate dihydrate,
14 mg/mL of propylene glycol,
5.5 mg/mL of phenol, and
2% w/w, relative to liraglutide, a stabilizing peptide that has the same amino acid sequence as liraglutide wherein said stabilizing peptide has a Ser¹⁴ to D-Ser¹⁴ substitution in the amino acid sequence.

20. A method for making the stabilized peptide composition according to claim 19, said method comprising: a) dissolving the sodium hydrogen phosphate dihydrate, propylene glycol and phenol in water to make Solution A; b) dissolving the liraglutide in water to make Solution B; c) dissolving the stabilizing peptide in water to make Solution C; d) combining Solution A, Solution B and Solution C while mixing to make the Combined Solution; and e) adjusting the pH of the Combined Solution to between 8.1 and 8.2 using either HCl(aq) or NaOH(aq).

21. The method according to claim 20 further comprising adding water to the Combined Solution.

22. The method according to claim 20, wherein said glucagon-like peptide and stabilizer are treated with base before preparing said stabilized peptide composition, wherein the stabilizer is a stabilizing peptide, wherein treating with base comprises: combining the glucagon-like peptide and stabilizing peptide in an aqueous solution of base, stirring or agitating the aqueous solution for from 10 minutes to 6 hours, and lyophilizing the aqueous solution comprising the glucagon-like peptide, stabilizing peptide and base.

23. The aqueous stabilized peptide composition according to claim 1, wherein the glucagon-like peptide and/or the stabilizer are treated with base before incorporation into the stabilized peptide composition.
